(19) United States
(12) Patent Application Publication
(10) Pub. No.: US 2009/0221834 A1
(43) Pub. Date: Sep. 3, 2009

(54) Synthesis of 5-Beta-Keto-1,2,4-Oxadiazoles and Conversion of 5-Beta-Keto-1,2,4 Oxadiazoles to N-Pyrazolyl Amidoximes

(76) Inventors: David R. Jensen, Harvest, AL (US); John E. Sidenstick, Newport, TN (US)

Correspondence Address: BUCHANAN, INGERSOLL & ROONEY PC, POST OFFICE BOX 1404, ALEXANDRIA, VA, 22313-1404, US

(21) Appl. No.: 12/226,484

(22) PCT Filed: Apr. 20, 2007
(86) PCT No.: PCT/US2007/009613

§ 371 Date: Mar. 26, 2009

Related U.S. Application Data
(60) Provisional application No. 60/793,249, filed on Apr. 20, 2006.

(51) Int. Cl. C07D 271/06; C07D 231/38

(52) U.S. Cl. 548/131; 548/371.7

(57) ABSTRACT

The disclosed invention relates to a process for preparing 5-β-keto-1,2,4-oxadiazoles of formula (I), and conversion of 5-β-keto-1,2,4-oxadiazoles (I) into N-pyrazolyl amidoximes of the formula (II) through reaction with hydrazine. The process is defined by two steps. An amidoxime, which may be prepared in situ, is condensed with a β-keto ester to form a 5-β-keto-1,2,4-oxadiazole. The 5-β-keto-1,2,4-oxadiazole is subsequently reacted with hydrazine to furnish the desired N-pyrazolyl amidoxime. The disclosed invention provides several advantages over the current state of the art for the synthesis of N-pyrazolyl amidoximes, which require the condensation of a pyrzolylamine with an actived substrate and subsequent reaction with hydroxyl amine. N-pyrazolyl amidoximes are useful synthetic intermediates, especially for the preparation of photographic developing chemicals.

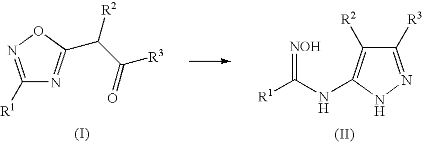

### BACKGROUND OF THE INVENTION

1. Field of the Invention

This invention relates to the efficient synthesis of 5-β-keto-1,2,4-oxadiazoles and the conversion 5-β-keto-1,2,4-oxadiazoles to N-pyrazolyl amidoximes via reaction with hydrazine. Both 1,2,4-oxadiazoles and N-pyrazolyl amidoxime are useful synthetic intermediates. Of particular note, N-pyrazolyl amidoximes are useful in the synthesis of the pyrazolo[1,5-b]1,2,4-triazole ring system, which is an important ring system in some photographic developing chemicals.

2. Brief Description of the Related Art

5-β-keto-1,2,4-oxadiazoles 1,2,4-Oxadiazoles have many known uses, including, but not limited to, anti-inflammatory and antiviral agents. The use of 1,2,4-oxadiazoles in pharmaceutical applications is attractive due to the desirable bioavailability and metabolic stability of many 1,2,4-oxadiazoles. Typically, 1,2,4-oxadiazoles are produced by a two-step reaction. The first step is the reaction of an amidoxime with an acylating agent such as an acid chloride or an acid anhydride to furnish an O-acyl amidoxime. The amidoxime is then typically reacted under strong base catalysis to effect cyclization and elimination of water, giving a 1,2,4-oxadiazole. However, the reaction can also be carried out under thermal conditions. Other methods to synthesize 1,2,4-oxadiazoles include the cycloaddition of an amidoxime with a nitrile oxide, the reaction of an amidoxime with diketene, and the reaction of an amidoxime with 2,2,6-trimethyl[1,3]dioxin-4-one. The later two reactions are of particular note as these methods deliver 5-β-keto-1,2,4-oxadiazoles.

Limitations to the typical synthesis of 1,2,4-oxadiazoles include the use of expensive and/or toxic bases such as NaH, NaOEt, pyridine, and TBAF. Additionally the use of an activated acid, such as an acid chloride or an acid anhydride limits the type of starting materials that can be used.

N-pyrazolyl amidoximes N-Pyrazolyl amidoximes are useful intermediates in the synthesis of various fine chemicals. Particularly, N-pyrazolyl amidoximes are useful in the synthesis of the pyrazolo[1,5-b]1,2,4-triazole ring system, which is an important ring system in some photographic developing chemicals. N-pyrazolyl amidoximes are typically synthesized by the reaction of an aminopyrazole with an imidate or imidoyl chloride to furnish an N-pyrazolyl amidine. The resulting N-pyrazolyl amidine is then reacted with hydroxylamine to furnish the desired N-pyrazolyl amidoxime. The reaction scheme is as follows:

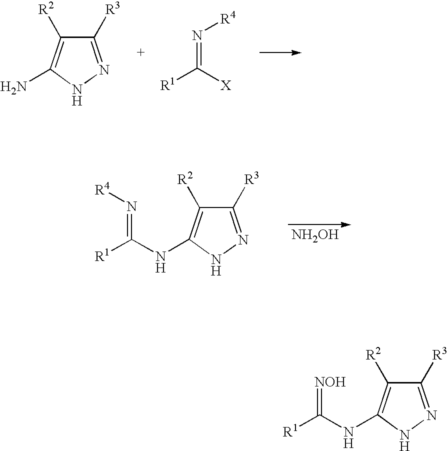

There are several limitations to the typical synthesis of N-pyrazolyl amidoximes. Aminopyrazoles are not generally commercially available and must be synthesized beforehand or prepared in situ. The typical aminopyrazole is formed by the reaction of hydrazine with a β-keto-nitrile, which are themselves often either not commercially available or relatively expensive. A second limitation to this method is the use of an activated carboxylic acid equivalent, such as an imidate, orthoester, or imidoyl chloride, which must be synthesized or formed in situ. Additionally, the use of these activated compounds can require the use of anhydrous conditions. Finally, the reaction of the N-pyrazolyl amidine with hydroxylamine is often problematic with respect to either purity or yield. This is of particular note as this is the final step of the synthesis. Some efforts have been made to circumvent some of these problems. For example, U.S. Pat. No. 6,020,498, and 6,555,711 use an alkyl or allyl group as R4, instead of a hydrogen atom. This allows a more expeditious route to the N-pyrazolyl amidoximes, but still has several limitations. For example, the both processes still requires the use of an activated carboxylic acid equivalent. Additionally the use of an alkyl or allyl group as R4 instead of hydrogen brings with it a number of problems including reduced throughput and increased waste of the process.

### SUMMARY OF THE INVENTION

The objective of the present invention is to develop an efficient process for making N-pyrazolyl amidoximes that avoids many of the shortcomings in the state of the art for making this class of important chemical intermediates. An important aspect as to how the present invention meets this objective is it avoids the need for an aminopyrazole as an intermediate. An additional objective for this invention is to describe a practical method for making 5-β-keto-1,2,4-oxadiazoles, which are used as intermediates in the synthesis of making N-pyrazolyl amidoximes.

The present invention relates a convenient synthesis of 5-β-keto-1,2,4-oxadiazoles and a novel process to convert a 5-β-keto-1,2,4-oxadiazole to a N-pyrazolyl amidoxime. The two key reactions can be carried out either separately or in a one-pot procedure. Isolation of the intermediate 5-β-keto-1,2,4-oxadiazole is preferred. The novel process described in this invention comprises the following two steps:

The process for making the 5-β-keto-1,2,4-oxadiazole comprises the reaction of an amidoxime of formula (III) with a β-keto-ester of formula (IV). The amidoxime is easily prepared by reaction of a nitrile with hydroxylamine and can either be isolated beforehand or prepared in situ. Additionally, the nitrile can be prepared by dehydration of an amide under standard conditions and can either be isolated beforehand or prepared in situ. In situ preparation of the amidoxime from the amide is the preferred method. The reaction is catalyzed by the presence of an appropriate base; examples of an appropriate base include sodium hydroxide, sodium methoxide, or potassium carbonate, with potassium carbonate being preferred. The base catalyst can be used in an amount ranging from 0.05 to 1.5 equivalents (i.e., molar ratio of from 0.05:1 to 1.5:1), based on the amount of the amidoxime. The reaction can be carried out in an organic solvent, such as acetonitrile, 2-propanol, or toluene. Alternatively, the reaction can be carried out in neat β-keto-ester (IV), with the neat reaction being preferred. The reaction is typically carried out at elevated temperatures, with 60-110° C. being preferred temperature range. The reaction can be carried out under atmospheric pressure, but this can lead to a build up by-products. When the reaction is carried out in a high boiling solvent, such as neat β-keto-ester (IV), the build up of by-products can be reduced by removing the alcohol and/or water that is formed during the reaction. This can be accomplished by use of a condenser column with cooling water set to a temperature where the solvent will be recondensed and the alcohol and/or water will remain in the gas phase and be removed. Once the reaction is judged complete, the 5-β-keto-1,2,4-oxadiazole can be isolated by standard methods, such as washing with water, removing solvents, filtration and/or drying. Alternatively if the reaction is carried out in an appropriate solvent the reaction mixture can be washed with water and used directly in conversion to an N-pyrazolyl amidoxime.

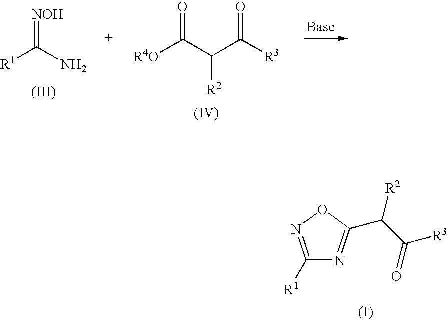

In the above formulae (I), (III) and (IV), R¹ represents an unsubstituted or substituted aromatic group, or an alkenyl group. The unsubstituted or substituted aromatic group preferably has 6 carbon atoms, and more preferably, has an electron-withdrawing substituent. The alkenyl group represented by R¹ preferably has 3 carbon atoms. R² represents H or an alkyl group, and preferably H. The alky group represented by R² preferably has 1 to 6 carbon atoms. R³ represents an alkyl group. The alky group represented by R³ preferably has 1 to 8 carbon atoms. R⁴ represents an alkyl group, and preferably a methyl group. The alkyl group represented by R⁴ preferably has 1 to 6 carbon atoms.

The process for converting a 5-β-keto-1,2,4-oxadiazole to an N-pyrazolyl amidoxime comprises reacting a 5-β-keto-1,2,4-oxadiazole of formula (I) with hydrazine or a salt thereof. Salts of hydrazine include, but are not limited to hydrazine hydrochloride. Hydrazine is preferably an aqueous solution of hydrazine. The 5-β-keto-1,2,4-oxadiazole for this reaction can be prepared by various methods, and can either be isolated beforehand or prepared in situ. The reaction is carried out in an organic solvent, preferably 2-propanol. The reaction is carried out at elevated temperatures, preferable 50-70° C. Additionally, an acid catalyst, preferably acetic acid, can be used to accelerate the reaction. When the reaction is complete, the N-pyrazolyl amidoxime can be isolated by standard methods, such as washing with water, removing solvents, filtration and/or drying.

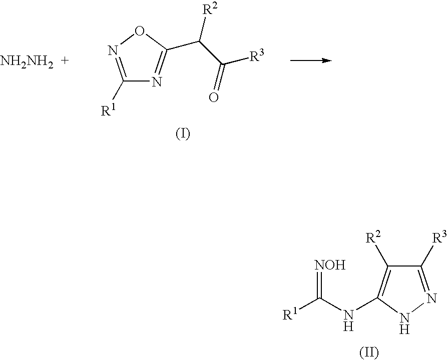

In the above formulae (II), R¹, R² and R³ have the same meanings as defined in formulae (I), (III) and (IV).

### EXAMPLE 1

### 3-(4-nitrophenyl)-5-(2-oxo-3,3-dimethyl butyl)-1,2,4-oxadiazole from 4-nitrobenzamidoxime

To a flask, 272.6 g of 4-nitrobenzamidoxime and 595.2 g of methyl pivaloylacetate are introduced. The reaction is setup with an efficient condenser with steady flow of 20-25° C. water. With the reaction temperature below 40° C., 208.3 g of potassium carbonate are added. The system is placed under vacuum at 40-60 mmHg. Once a consistent vacuum of 40-60 mmHg is achieved, the reaction mixture is heated to about 85° C. and held at this temperature and pressure. The reaction should last about 12 hours and can be monitored by HPLC.

Once the reaction is complete, the temperature of the reaction mixture is adjusted to 70° C. and 500 mL of water is added. The lower aqueous phase is removed while the reaction mixture is kept between 65-75° C. Maintaining the reaction mixture at 70° C., 50.4 g of acetic acid is added. The reaction mixture is cooled until crystals begin to form and is held at that temperature for about an hour. The reaction mixture is cooled to 0-5° C. at a rate of 20° C. per hour. The desired 5-β-keto-1,2,4-oxadiazole is isolated on a Buchner funnel and washed with 0° C. methanol. The product is dried in a vacuum oven to give 348 (80% yield) of the desired 5-β-keto-1,2,4-oxadiazole.

### EXAMPLE 2

### 3-(4-nitrophenyl)-5-(2-oxo-3,3-dimethyl butyl)-1,2,4-oxadiazole from 4-nitrobenzonitrile

To a flask, 222.9 g of 4-nitrobenzonitrile is added. To this, 593 g of methanol and 195 g of toluene are added. The mixture is heated to 60° C. To this 104.5 g of a 50% aqueous solution of hydroxylamine is added over 15 minutes. The reaction is monitored by HPLC and continued until there is less than 0.5% remaining 4-nitrobenzonitrile. The solvent is removed by vacuum distillation.

To the resulting yellow solid, 595.2 g of methyl pivaloylacetate is added. The reaction flask is fitted with an efficient condenser with steady flow of 20-25° C. water. The mixture is refluxed under vacuum (55-60 mmHg), until the temperature of the mixture is greater than 85° C. During this step any residual methanol, toluene, water, or hydroxylamine should be removed, and the methyl pivaloylacetate should be recondensed and returned to the reaction mixture. With the reaction temperature below 40° C., 208.3 g of potassium carbonate are added. The system is placed under vacuum at 40-60 mmHg. Once a consistent vacuum of 40-60 mmHg is achieved, the reaction mixture is heated to about 85° C. and held at this temperature and pressure. The reaction should last about 12 hours and can be monitored by HPLC.

Once the reaction is complete, the temperature of the reaction mixture is adjusted to 70° C. and 500 mL of water is added. The lower aqueous phase is removed while the reaction mixture is kept between 65-75° C. Maintaining the reaction mixture at 70° C., 50.4 g of acetic acid is added. The reaction mixture is cooled until crystals begin to form and is held at that temperature for about an hour. The reaction mixture is cooled to 0-5° C. at a rate of 20° C. per hour. The desired 5-β-keto-1,2,4-oxadiazole is isolated on a Buchner funnel and washed with 0° C. methanol. The product is dried in a vacuum oven to give 348 g (80% yield) of the desired 5-β-keto-1,2,4-oxadiazole.

### EXAMPLE 3

### 3-(4-nitrophenyl)-5-(2-oxo-3,3-dimethyl butyl)-1,2,4-oxadiazole from 4-nitrobenzamide

To a flask, 250 g of 4-nitrobenzamide is added. To this 1300 g of toluene and 212.4 g of N,N-dimethyl formamide are added. The resulting mixture is agitated and heated to 50° C. To this mixture, 171.6 g of phosphorus oxychloride is added over 10-15 minutes, during which time an approximately 20° C. exotherm is observed. After 30 minutes, 500 g of water is charged maintaining the temperature of the reaction mixture between 50-70° C. The reaction mixture is held at 50-60° C. for 30 minutes without agitation. The lower aqueous phase is removed. The resulting solution is concentrated by vacuum distillation until 70-90% of the toluene has been removed.

To this slurry, 593 g of methanol is added. The mixture is heated to 60° C. To this 104.5 g of a 50% aqueous solution of hydroxylamine is added over 15 minutes. The reaction is monitored by HPLC and continued until there is less than 0.5% remaining 4-nitrobenzonitrile. The solvent is removed by vacuum distillation.

To the resulting yellow solid, 595.2 g of methyl pivaloylacetate is added. The reaction flask is fitted with an efficient condenser with steady flow of 20-25° C. water. The mixture is refluxed under vacuum (55-60 mmHg), until the temperature of the mixture is greater than 85° C. During this step any residual methanol, toluene, water, or hydroxylamine should be removed, and the methyl pivaloylacetate should be recondensed and returned to the reaction mixture. With the reaction temperature below 40° C., 208.3 g of potassium carbonate are added. The system is placed under vacuum at 40-60 mmHg. Once a consistent vacuum of 40-60 mmHg is achieved, the reaction mixture is heated to about 85° C. and held at this temperature and pressure. The reaction should last about 12 hours and can be monitored by HPLC.

Once the reaction is complete, the temperature of the reaction mixture is adjusted to 70° C. and 500 mL of water is added. The lower aqueous phase is removed while the reaction mixture is kept between 65-75° C. Maintaining the reaction mixture at 70° C., 50.4 g of acetic acid is added. The reaction mixture is cooled until crystals begin to form and is held at that temperature for about an hour. The reaction mixture is cooled to 0-5° C. at a rate of 20° C. per hour. The desired 5-β-keto-1,2,4-oxadiazole is isolated on a Buchner funnel and washed with 0° C. methanol. The product is dried in a vacuum oven to give 348.25 g (80% yield) of the desired 5-β-keto-1,2,4-oxadiazole.

### EXAMPLE 4

### Preparation of N-(3-tert-butyl-5-pyrazolyl)-4-nitrobenzaime oxime

To a 500 mL flask, 100 g of 3-(4-nitrophenyl)-5-(2-oxo-3,3-dimethyl butyl)-1,2,4-oxadiazole is added. To this 159 g of 2-propanol, and 5.18 g of acetic acid are added. The contents of the flask are heated to 55-60° C. To this mixture, 30.8 g of a 54% solution of hydrazine in water is added over about 15 minutes. After the hydrazine addition is complete, the reaction mixture is held at 55-60° C. for about 4 hours, or until there is less than 1% remaining starting material as judged by HPLC. The reaction mixture is cooled to 55° C. 11.1 g of water is added. The reaction is seeded with N-(3-tert-butyl-5-pyrazolyl)-4-nitrobenzamide oxime, and cooled to 25° C. at a rate of 30° C./hour. With the reaction at 25° C., 148.2 g of H₂O is added and held at this temperature for 2 hours. The reaction mixture is cooled to 5° C. at a rate of 10° C./hour. The reaction mixture is held for an additional hour at 0-5° C. The product is isolated by filtration on a Buchner funnel and is washed with a 0-5° C. solution of 45% 2-propanol in water. Drying the product in a vacuum oven gives 92.27 g (88.0% yield) of the desired with N-(3-tert-butyl-5-pyrazolyl)-4-nitrobenzamide oxime.

What is claimed is:

1. A process for preparing a 5-β-1,2,4-oxadiazole comprising reacting an amidoxime with a β-keto-ester.

2. The process of claim 1, wherein the reaction is carried out in the presence of a base catalyst.

3. The process of claim 2, wherein the base catalyst is at least one selected from the group consisting of sodium hydroxide, sodium methoxide and potassium carbonate.

4. The process of claim 3, wherein the base catalyst comprises potassium carbonate.

5. The process of claim 2, wherein the base catalyst/amidoxime molar ratio is from 0.05:1 to 1.5:1.

6. The process of claim 5, wherein the base catalyst/amidoxime molar ratio is about 1:1.

7. The process of claim 1, wherein the reaction is carried out in the absence of a solvent.

8. The process of claim 7, which further comprising simultaneously removing the alcohol and water formed in the reaction.

9. The process of claim 1, wherein the reaction is carried out in the presence of an organic solvent.

10. The process of claim 9, wherein the organic solvent is at least one selected from the group consisting of acetonitrile, 2-propanol and toluene.

11. The process of claim 1, wherein the reaction is carried out at a temperature ranging from 60° C. to 110° C.

12. A process for preparing a N-pyrazolyl amidoxime comprising reacting a 5-β-1,2,4-oxadiazole with hydrazine or a salt thereof.

13. The process of claim 12, wherein the hydrazine is an aqueous solution of hydrazine.

14. The process of claim 13, wherein the reaction is carried out in a solvent.

15. The process of claim 14, wherein the solvent comprises 2-propanol.

16. The process of claim 12, wherein the reaction is carried out at a temperature ranging from 50° C. to 70° C.

17. The process of claim 12, wherein the reaction is carried out in the presence of an acid catalyst.

18. The process of claim 17, wherein the acid catalyst comprises acetic acid.

19. A process for preparing a N-pyrazolyl amidoxime comprising: (i) reacting an amidoxime with a β-keto-ester to prepare a 5-β-1,2,4-oxadiazole; and (ii) reacting the 5-β-1,2,4-oxadiazole with hydrazine.

20. The process of claim 19, which further comprising isolating the 5-β-1,2,4-oxadiazole obtained in step (i), prior to step (ii).
